(19) United States
(12) Patent Application Publication
(10) Pub. No.: US 2023/0248940 A1
(43) Pub. Date: Aug. 10, 2023

(54) Neurovascular Aspiration Catheter with Shaped Tip

(71) Applicant: Toro Neurovascular, Inc., (US)

(72) Inventor: Hyung Posalit, Dana Point, CA (US)

(73) Assignee: Toro Neurovascular, Inc., Dana Point, CA (US)

(21) Appl. No.: 18/104,694

(22) Filed: Feb. 1, 2023

Related U.S. Application Data
(60) Provisional application No. 63/307,150, filed on Feb. 6, 2022.

(51) Int. Cl. A61M 25/00; A61M 25/01

(52) U.S. Cl. CPC A61M 25/005; A61M 25/0108; A61B 2017/22079

(57) ABSTRACT

An aspiration catheter has an elongate flexible tubular body that has a side wall and a distal tip. A helical coil is embedded in the side wall and extends from the proximal end of the catheter to a location offset proximally from the distal tip. A reinforcement fiber is longitudinally embedded in the side wall starting from a location offset proximally from the distal tip and extends proximally at least 10 cm. The distal tip has a shaped edge which assists the catheter tip in navigating past ledges in bifurcations.

### BACKGROUND OF THE INVENTION

### 1. Field of the Invention

The present invention relates to a neurovascular aspiration catheter with a shaped tip that can be used to perform direct aspiration thrombectomy in patients experiencing large vessel occlusions.

### 2. Description of the Prior Art

Large vessel occlusions in the cerebrovasculature cause ischemic strokes. Stroke remains a largely unaddressed problem that affects over 800,000 Americans each year. The number of deaths due to stroke continues to increase. Treatment technologies have significantly improved as thrombectomy designs have become more refined, but the need for improved effectiveness and efficiency remains.

In recent years several clinical trials established mechanical thrombectomy via the use of a stent-retriever as the gold-standard treatment option in patients with large vessel occlusions. Even more recent data suggest that direct aspiration via the use of a large-bore catheter is a viable alternative to stent retriever-based methods. Direct aspiration thrombectomy uses an aspiration catheter with a large inner lumen (large bore) to aspirate and remove the thrombus. If aspiration is not successful, the large bore aspiration catheter can serve as a platform to deliver a stent retriever or other devices.

Conventional large bore aspiration catheters suffer from a few drawbacks.

First, almost all large bore aspiration catheters on the market today have the same straight-tip design: a flat edge that is perpendicular to the central axis of the catheter. A common issue with large-bore aspiration catheters is the inability to navigate the catheter through complex and tortuous neuro-vasculature to reach the site of the occlusion. One specific challenge during navigation is getting the tip of the catheter to move past certain junctions within intracranial arteries, such as the origin of the ophthalmic artery. The tip of a catheter can become entrenched or stuck in these types of locations, a phenomenon known as the “ledge effect.” This is best illustrated in FIGS. 1A and 1B, where FIG. 1A shows the distal end of an aspiration catheter C tracked over a guidewire G approaching a ledge L, and FIG. 1B showing the distal tip of the catheter C being stuck at the ledge L. The straight-tip profile makes for a challenging condition when tracking over a guidewire and approximately half or more of the tip cross-section is against the ledge L and leaning into the bifurcation. This occurs because there is a large difference between the diameter of the guidewire and the inner diameter of the catheter, so that the guidewire is pressed against the side wall of the catheter and far from the center-line. Additional pushing and pulling of the catheter C and guidewire are often insufficient to alleviate this situation. Thus, such large bore catheters with a straight-tip configuration may not be effective in navigating past these locations, as once the distal tip hits the ledge it is difficult to advance the catheter forward and out of the ledge.

Second, one existing risk of current aspiration catheters is a tensile break at the distal tip of the catheter. As players in the neurovascular space seek to increase aspiration catheters in size, the major design challenge companies faced has been to enable the catheters to remain soft enough to navigate the tortuous anatomy (significant twists and turns) of the arteries in the brain despite the catheter being significantly larger and thus having more total material. In pursuit of this, companies have used increasingly softer and weaker polymer materials in the design of the distal portion of catheters, thus sacrificing tensile strength. In an effort to make catheters more flexible and navigable, some designs have resulted in low tensile strength at the distal ends of the catheter. These catheters with low tensile strength are at increased risk of breaking and material separation during use, which can lead to clinical harm and adverse events.

Additionally, a larger catheter results in increased contact surface against blood vessel walls which causes an increase in friction force. Paired with this, the increased diameter in a user's hand likely leads to the perception of a more robust product that can withstand greater forces. That is, a physician is likely to apply more force when pulling a larger catheter if it sticks than they may apply to a smaller catheter.

The combination of these factors has resulted in various aspiration catheter products with low tensile strength, leading to broken catheters, undesirable outcomes, and adverse events when used in the field.

Thus, there remains a need for a large bore aspiration catheter that overcomes these drawbacks.

### SUMMARY OF THE DISCLOSURE

It is an object of the present invention to provide a large bore aspiration catheter that has a shaped tip that would provide improved ease of navigation, especially past ledges in the vasculature.

It is another object of the present invention to provide a large bore aspiration catheter that has improved distal tensile strength that would result in fewer procedures with adverse events due to tensile failures.

In order to accomplish the objects of the present invention, there is provided an aspiration catheter having an elongate flexible tubular body that has a side wall and a distal tip. A helical coil is embedded in the side wall and extends from the proximal end of the catheter to a location offset proximally from the distal tip. A reinforcement fiber is longitudinally embedded in the side wall starting from a location offset proximally from the distal tip and extends proximally at least 10 cm. The distal tip has a shaped edge which assists the catheter tip in navigating past ledges in bifurcations.

In one embodiment, the distal tip has a wave-shaped edge which smoothly transitions between peaks and valleys circumferentially to define tapers.

### BRIEF DESCRIPTION OF THE DRAWINGS

FIGS. 1A and 1B illustrate how the distal tip of a conventional aspiration catheter can become entrenched or stuck in junctions within intracranial arteries.

FIG. 2 is a perspective view of an aspiration catheter according to one embodiment of the present invention.

FIG. 3 is a side view of the catheter of FIG. 2.

FIG. 4 is a top view of the catheter of FIG. 2.

FIG. 5 is a cross-sectional side view of the distal zone of the catheter of FIG. 3.

FIG. 6 is a cross-sectional view taken along line 6-6 of FIG. 5.

FIGS. 7A and 7B are two different isometric views of the distal port of the catheter of FIG. 3.

FIGS. 8A-8C illustrate how the distal wave tip of the catheter of FIG. 2 assists the catheter in navigating past a ledge L in a vessel bifurcation.

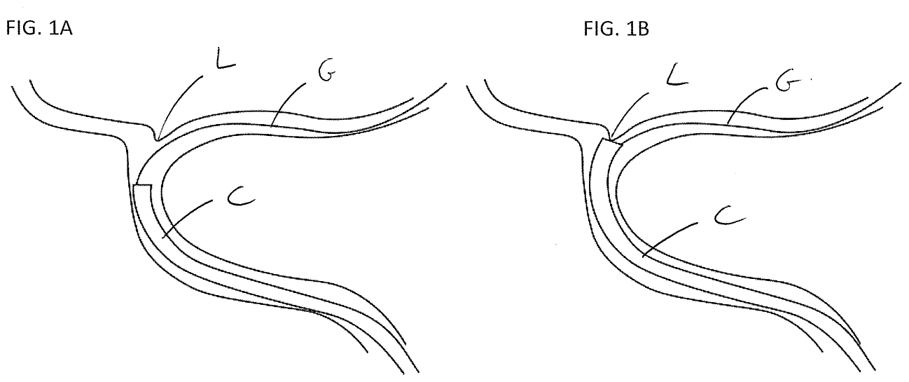

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

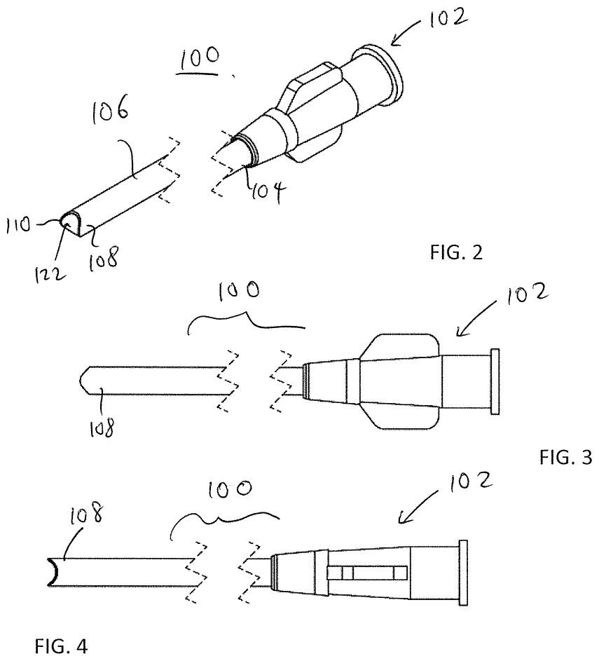

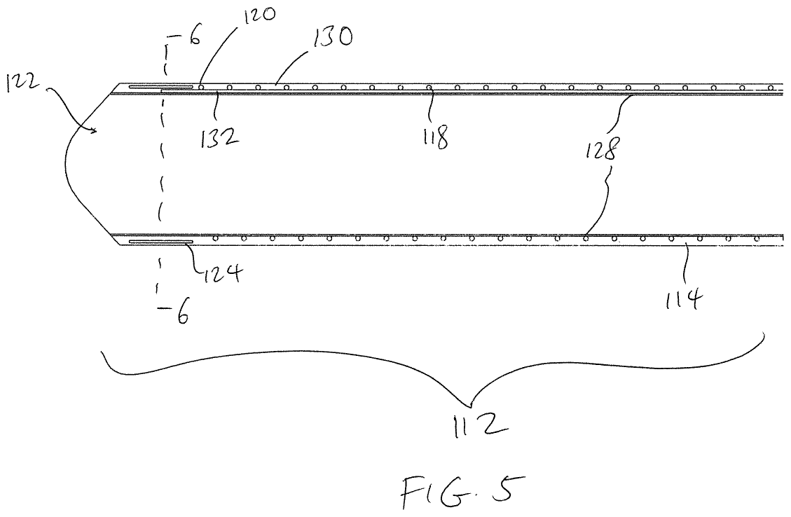

FIGS. 2-6 illustrate one embodiment of an aspiration catheter 100 of the present invention having a distal wave tip. Referring to FIGS. 2-6, the aspiration catheter 100 has a handle assembly 102 at a proximal end 104 thereof, and an elongate flexible tubular body 106 extending from the handle assembly 102 to a distal end 108. The distal end 108 has a novel distal wave tip 110. The tubular body 106 has a side wall 114 with an optional inner liner 128 that defines a central lumen 116, the central lumen 116 having a longitudinal axis. An outer jacket 130 defines the outer layer or skin of the side wall 114. The side wall 114 is itself composed of the inner liner 128 and the outer jacket 130. The side wall 114 can alternatively just be composed of a jacket material (i.e., the liners are optional), so that the jacket could define both the inner and outer surface of the side wall 114.

The handle assembly 102 includes a hub that is attached to the proximal end 104 of the catheter 100 with adhesive. The inner lumen (not shown) of the hub matches up with the proximal port of the tubular body 106. The inner lumen of the hub flares into a conical shape which acts as a “funnel” into the tubular body 106 of the catheter 100. The hub is made of a single polymer material such as nylon, polycarbonate, or a similar rigid material.

The inner liner 128 is a single-material, thin, tube which acts as the exposed contact surface in the inside of the central lumen 116 and spans the entire catheter length from proximal to distal end. The inner liner 128 can also be composed of multiple materials stacked in tight layers, known as “tie layers” as the intermediate layers that serve to act as a bonding bridge between the inner liner and outer jacket. It is preferably made of a low friction material, such as PTFE, polyolefins (which include HDPE and LDPE), or blended polymeric materials (e.g., there are compounds which contain traditional polymers mixed with lubricious additives) which may be created by extrusion or by dip coating onto a mandrel. The liner tubing has a wall thickness that may range from 0.0001″ to 0.003″.

The tubular body 106 has a distal port 122 at the distal wave tip 110, and a distal zone 112. The distal port 122 may have its edges rounded or tapered or chamfered in order to be atraumatic.

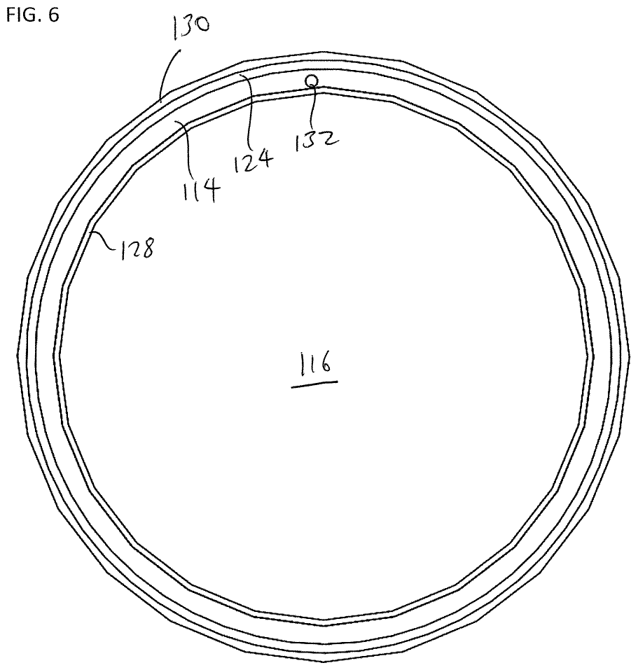

The distal zone 112 is illustrated in greater detail in FIGS. 5 and 6 and has a helical coil 118 embedded in the side wall 114 of the tubular body 106, the helical coil 118 having a distal end 120 that is spaced apart from the distal port 122 of the tubular body 106. A tubular radiopaque marker 124 is embedded in the side wall 114 at a location between the distal end 120 of the helical coil 118 and the distal port 122 of the tubular body 106.

The helical coil 118 is created by wrapping one or more strands of wire around the inner liner 128 from the distal end 108 to the proximal end 104. The coil wire may be Stainless Steel, Nitinol, or another appropriate metal or polymer dependent on design considerations. The coil wire may be circular, rectangular, or ovular in cross-sectional shape. The coil wire may be between 0.0003″ and 0.009″ in cross-sectional dimensions.

The radiopaque marker 124 may comprise any of a variety of radiopaque materials, such as a platinum/iridium alloy, with a wall thickness between 0.0005″ and 0.003″. The radiopaque marker 124 can be a broken or continuous band that is placed over the distal end of the inner liner 128 and located at the distal end of the helical coil 118.

One or more reinforcement fibers 132 can be longitudinally embedded in the side wall 114 at the distal zone 112, and can be bonded to the tubular radiopaque marker 124. The reinforcement fiber 132 is one or more strand(s) of material oriented longitudinally along the catheter 100 and pinned between the inner liner 128 and the helical coil 118. One end of the reinforcement fiber 132 is attached at the marker 124 near the distal end 108 of the catheter 100, and the reinforcement fiber 132 runs in the proximal direction along the catheter 100 by at least 10 cm but may run up to the entire catheter length dependent on design considerations. Reinforcement fibers 132 can be mono- or multi-filament and are made from high mechanical strength materials, including but not limited to carbon fiber, liquid crystal polymer (LCP), carbon fiber, Kevlar, and other synthetic fibers. The reinforcement fibers 132 can range in diameter from 0.0001″ to 0.010″.

One or more reinforcement fibers 132 can be provided; even though FIGS. 5 and 6 illustrate one reinforcement fiber 132, additional reinforcement fibers 132 can be provided in a bundle, or dispersed circumferentially around the tubular body 106. The reinforcement fibers 132 can extend longitudinally or may be marginally coiled along the length of the tubular body 106.

The outer jacket 130 is the outer surface of the side wall 114 which is comprised of varying polymer materials, such as Nylon, Pebax, polyether-based thermoplastic urethanes (TPU), polyester-based TPUs, polycarbonate-based TPUs, and others. Each outer jacket material begins as a single tube of material (i.e., an “extrusion” or “single lumen extrusion”) which is placed over the inner liner 128, reinforcement fiber 132, helical coil 118 and marker 124 configuration. The stiffer polymer materials are placed on the proximal end 104 of the catheter 100 while the less stiff polymers are placed distally, ensuring that the catheter stiffness decreases from the proximal end 104 to the distal end 108. Each jacket material is loaded end-to-end against the neighboring jacket material. Heat is then applied to the entire assembly, with use of processing aids such as FEP heatshrink, to allow the jacket polymers to melt and flow into and embed the helical coil 118 and the marker 124 to create a single composite assembly. FEP heatshrink is a material that is placed over the entire catheter assembly during manufacturing, and as it is heated, it gets hot and pinches down, which holds all of the catheter jacket materials together as they flow.

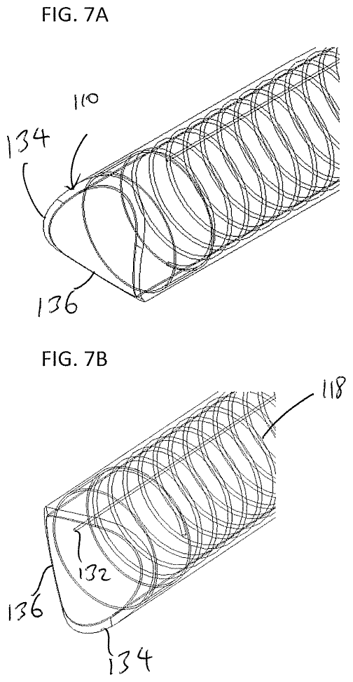

In the embodiment of FIGS. 2-6, the distal port 122 comprises a distal wave tip 110 with a wave-shaped edge which smoothly transitions between peaks 134 and valleys 136 circumferentially to define tapers. There may be as many as one peak 134 and one valley 136 and up to five peaks 134 and five valleys 136. The distance between each peak 134 and valley 136 may be as small as 0.1 mm and as large as 3 mm. FIGS. 7A and 7B are two different isometric views of the distal port 122 of the catheter of FIG. 3 showing these peaks 134 and the valleys 136.

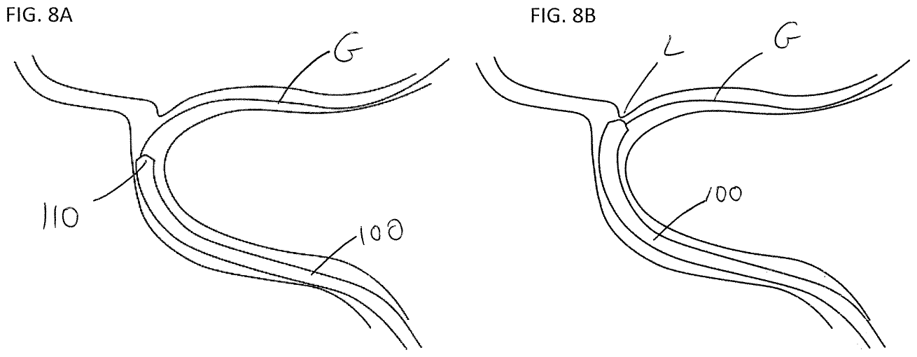

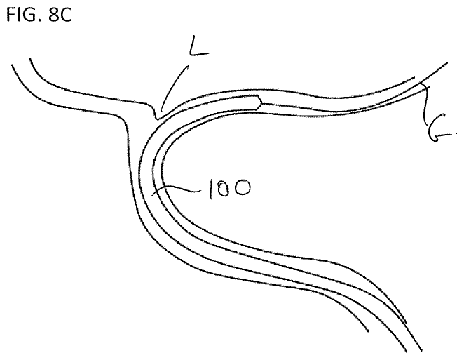

FIGS. 8A-8C illustrate how the distal wave tip 110 assists the catheter 100 in navigating past a ledge L in a vessel bifurcation. Initial access to the site of an occlusion is typically achieved by utilizing a guidewire G and/or a microcatheter to navigate through tortuosity of the neuro-vasculature to a blocked artery. Once the guidewire and/or microcatheter are in place, an aspiration catheter 100 can be advanced over the guidewire and/or microcatheter, with the guidewire and/or microcatheter inside of the central lumen 116 of the aspiration catheter 100. In FIG. 8A, the catheter 100 is tracked over a guidewire G. In FIG. 8B, the distal end 108 of the catheter 100 engages the ledge L. The wave shape of the distal wave tip 110 helps to alleviate this situation as it creates a taper which helps to prevent the distal wave tip 110 from getting stuck at the ledge L. Depending on the orientation of the catheter 100 when it reaches the ledge L, a quarter-turn of the catheter 100 may be all that is needed to enable the distal wave tip 110 to disengage the ledge L, and the taper on the distal wave tip 110 helps the catheter 100 pass through, as shown in FIG. 8C. In some situations, the peaks 134 and valleys 136 allow simple passage past the ledge L without the need for even turning the catheter 100.

Thus, the aspiration catheter 100 with a shaped tip can be used by physicians during endovascular treatment of acute ischemic stroke. The catheter tip has an edge that is wavy, as opposed to traditional catheter tips which are straight. The catheter also has increased tensile strength due to reinforcement of the distal end, while not compromising navigability and flexibility.

The reinforcement fiber 132 extends along the distal length to provide composite support, much in the same way that rebar supports concrete. Any tensile applied to the catheter is directly supported by the reinforcement fiber 132. Since it is a thin fiber, it does not add significant stiffness to the bending profile of the distal end of the catheter, so it allows the catheter to achieve bending properties necessary for navigating the tortuous anatomy of the neurovasculature while also maintaining superior tensile strength.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. An aspiration catheter, comprising:
an elongate flexible tubular body having a proximal end, a side wall and a distal tip, wherein a helical coil is embedded in the side wall and extends from the proximal end to a location offset proximally from the distal tip, a reinforcement fiber is longitudinally embedded in the side wall starting from a location offset proximally from the distal tip and extending proximally at least 10 cm; and
wherein the distal tip has a wave-shaped edge which smoothly transitions between peaks and valleys circumferentially to define tapers.

2. The catheter of claim 1, wherein there are one to five peaks and one to five valleys.

3. The catheter of claim 2, wherein the distance between each peak and each valley ranges from 0.1 mm to 3 mm.

4. The catheter of claim 1, wherein the helical coil has a distal end, and further including a radiopaque marker band that is proximal of the distal tip and at the distal end of the helical coil.

5. The catheter of claim 4, wherein one end of the reinforcement fiber is attached at the radiopaque marker band.
